(19) United States
(12) Patent Application Publication
(10) Pub. No.: US 2016/0113746 A1
(43) Pub. Date: Apr. 28, 2016

(54) LIGHT SOURCE, DETECTOR AND LUMINESCENT COMPOSITE

(71) Applicant: Joseph F. Bringley, Rochester, NY (US)

(72) Inventor: Joseph F. Bringley, Rochester, NY (US)

(21) Appl. No.: 14/923,435

(22) Filed: Oct. 27, 2015

Related U.S. Application Data
(60) Provisional application No. 62/069,358, filed on Oct. 28, 2014.

(51) Int. Cl. A61C 13/15; A61K 6/083; A61K 6/00

(52) U.S. Cl. CPC A61C 19/004; A61K 6/002; A61K 6/0088; A61K 6/0052; A61K 6/083

(57) ABSTRACT

The present invention relates to a system comprising photopolymerizable composites and an associated light polymerization device. The composite comprises a photopolymerizable resin and a luminophore, and optionally inorganic fillers and a photobleaching agent. The polymerization device comprises a light source and a photodetector. The composite and the device are coupled via a feedback loop to indicate the degree of polymerization, or to indicate that polymerization is complete. The invention relates to any photopolymerizable composite or coating including industrial, protective, biomedical and dental composites and coatings.

### CROSS REFERENCE TO RELATED APPLICATIONS

Priority is claimed and reference is made to commonly assigned, U.S. Prov. Patent Application Ser. No. 62/069,358 by Bringley et al., filed Oct. 28, 2014 entitled “LIGHT SOURCE, DETECTOR AND LUMINESCENT COMPOSITE”, the disclosures of which are incorporated herein by reference.

### FIELD OF THE INVENTION

The present invention relates to photopolymerizable composites and an associated light polymerization device. The composite comprises a photopolymerizable resin and a luminophore, and optionally inorganic fillers and a photobleaching agent. The polymerization device comprises a light source and a photodetector. The composite and the device are coupled via a feedback loop to indicate the degree of polymerization, or to indicate that polymerization is complete. The invention relates to any photopolymerizable composite or coating including industrial, protective, biomedical and dental composites and coatings.

### BACKGROUND OF THE INVENTION

Inorganic-organic polymer composite materials are used in a wide variety of applications including structural materials, high performance composites, optical components, aerospace, biomedical implants and dental applications. Generally, composites are employed where performance requirements are demanding and not easily fulfilled with traditional structural materials. For example, inorganic materials such as glass, ceramic and stone are very hard, scratch resistant and even sometimes transparent (e.g., glass) but suffer from the fact that they are very heavy and brittle. Polymers, conversely, are light and durable but have poor hardness, abrasion and wear resistance.

Composites, made from the combination of inorganic materials and polymers, may have properties that lie in between, potentially providing materials that are simultaneously strong but lightweight, hard but flexible, abrasion resistant and durable. In order to achieve such properties, in practice, hard inorganic materials are mixed into polymers, or polymer precursors, monomers and/or oligomers, (hereafter collectively referred to as resins) and the mixture is then cured to form a composite. In recent years, the polymer industry is transforming from composites that are polymerized, or “cured”, using heat (thermal set polymers) to composites that are cured using ultraviolet or visible light, or low energy electrons (hereafter called UVEB resins).

UVEB curable resins offer tremendous energy and waste savings to the coatings and composites industries because they are polymerized (cured) directly with light or low energy electrons, and also because they generally do not contain volatile diluents such as solvents or carriers that may be considered hazardous air pollutants. UVEB curing is far more energy efficient since it overcomes the thermal loss that is prevalent in conventional thermoset coating systems. Ironically, the fundamental advantages of UVEB systems, where a solventless medium is cured rapidly by radiation, are also the source of significant system limitations.

Light curing requires that the coating and/or object must be sufficiently transparent in the spectral region of curing, since the penetration depth and absorption of the curing radiation is essential to achieve rapid and efficient curing. This limits the performance additives (fillers, stabilizers, functional additives, and coating aids) that can be added to UVEB systems since the additives must also fulfill the requirement of being sufficiently transparent in the curing region of the spectrum.

Furthermore, in thick coatings or composites, the degree of curing may vary across the specimen due to the attenuation and absorption of curing radiation. To overcome this problem, in practice, it is common to “overexpose” the specimen with curing radiation in order to assure that curing is complete or near complete. This is not ideal since energy and time are thereby wasted. Furthermore, in medical applications, such as dentistry, overexposure may increase risk to the patient.

The dental industry, primarily due to health concerns, is rapidly transitioning dental restoratives (e.g., cavity fillings, dental restorations, adhesives, etc.) from the conventional mercury based amalgams to highly filled, light curable, resin based composites. Resin based composites are safer and better match the color and appearance of human tooth enamel, but are often softer, not as strong or as durable as the traditional metal amalgams. To resolve these problems, manufacturers have developed micro filled polymer composites that have strength, hardness and durability close to that of the conventional amalgams. Typically the resin based composite paste is applied or packed into a tooth cavity and then cured using a hand-held light wand. The light wand is held in proximity of the composite for a period of time believed necessary to fully cure the paste with the intention to create a hard, strong and durable composite.

There is a significant clinical problem, however, in that inadequate curing can lead to premature failure of the composite requiring clinical revision of the restoration and significant patient cost. The extent and significance of the problem has been carefully described in recent dental publications including “Light-Curing Units: A Review of What We Need to Know”, Price et al. Journ. Dental Res. (2015), and “Light-curing of resin based composites in the LED era”, Kramer et al. American Journ. Dentistry (2008), and are incorporated herein by reference. The cure rate and cure depth of a restoration is dependent upon a number of factors including the composite thickness, composite color, light absorption and attenuation of light within the composite. This is further compounded by the variability in lamp designs and power outputs of lamps from various manufacturers, and the degradation of the lamp over time, and yet even further complicated by user variability in terms of how far the lamp tip is held from the composite and for how long the composite is irradiated with polymerizing light. Today, a dentist may follow manufacturer's guidelines, but still has no method of determining if the restoration was sufficiently cured.

PCT WO 2011/140469 to Fathi et al., incorporated herein by reference in its entirety, discloses a polymerizable composition including at least one monomer, a photoinitiator capable of initiating polymerization of the monomer when exposed to light, and a phosphor capable of producing light when exposed to radiation (typically X-rays). The material is particularly suitable for bonding components at ambient temperature in situations where the bond joint is not accessible to an external light source.” There is a problem, however, in that the invention is directed toward curing (with X-rays) opaque structures that are not accessible to UV or visible light. There is an additional problem in that the invention does not include a detector system capable of indicating that polymerization is substantially complete.

### Problem to be Solved

The inventors have recognized that there is a problem in that there is, currently, no method of indicating the degree of polymerization within a composite in real time and that both under-cured and overexposed composites may directly result. Under-cured composites may lack strength and suffer with respect to mechanical properties, whereas overexposure wastes time and energy. There is a need for a composite/device system that may indicate whether the proper exposure of curing radiation has been applied to the composite.

### SUMMARY OF THE INVENTION

The present invention relates to a device-composite system comprising a light curable resin and a luminophore capable of absorbing a first wavelength of light and producing light of a second wavelength, and a curing lamp comprising a light source producing said first wavelength of light and a detector capable of detecting said second wavelength of light to produce a signal The invention also provides a dental device-composite system comprising a light curable dental composite comprising a resin, at least one luminophore capable of absorbing a first wavelength of light and producing light of a second wavelength, and one or more material selected from the group consisting of fillers, optical brighteners, and photoinitiators, and a dental curing lamp comprising a light source of a first wavelength and a detector capable of detecting said second wavelength of light, wherein the light of a second wavelength is detected by the detector as close as possible to the light exit position of said dental lamp.

### Advantageous Effect of the Invention

The invention provides a simple and reliable method that provides, in real time, direct information regarding the amount of incident curing radiation impinging upon a photopolymerizable article. The information can be used to determine the degree of photocure and/or determine if the cure has been complete. In dentistry, the invention provides for better quality teeth restorations that have greater durability, leading to better patient outcomes.

### BRIEF DESCRIPTION OF THE DRAWINGS

FIG. 1 represents a component (the composite component) of the invention. The composite component contains at minimum a light curable resin and a luminophore.

FIG. 2 represents a second component (the detector component) of the invention. The curing lamp includes a detector capable of detecting the emission of the luminophore.

FIG. 3 represents the luminescence signal versus time (upper curves) for specimens of 5 mm diameter and thicknesses (a) 1 mm, (b) 2 mm and (c) 3 mm, respectively. The lower curve represents the instantaneous slope of the emission.

FIG. 4 represents the luminescence signal versus time (upper curve) for a specimen of 10 mm diameter and 2 mm thickness. The lower curve represents the instantaneous slope of the emission.

FIG. 5 represents the luminescence signal versus time (upper curves) for (a) an unshaded commercial composite containing 530 ppm of a near infrared phosphor, and (b) a dark shade commercial composite containing 526 ppm of a near infrared phosphor. The lower curve represents the instantaneous slope of the emission.

### DETAILED DESCRIPTION OF THE INVENTION

The invention provides a composite and device that are coupled via a feedback loop to indicate the degree of polymerization, or to indicate that polymerization, interchangeably referred to herein as curing, is complete. The composite comprises a light polymerizable/curable resin and a luminophore, most preferably a phosphor, and optionally inorganic fillers and a photobleaching agent. The polymerization device comprises a light source, an optional long pass light filter, and a photodetector. The luminophore, most preferably a phosphor, is selected so that it absorbs a fraction of the curing radiation from the light source and emits light of a second wavelength. The emitted light is detected and integrated by the photodetector. Since the amount of light emitted by the luminophore, most preferably a phosphor, is directly proportional to the amount of incident (curing) radiation, the signal detected by the photodetector is a real time measure of the total flux (time×intensity) of curing radiation absorbed by the specimen, and therefore can be related, or calibrated to, the degree of polymerization or curing of the composite. In a particular embodiment, the coating or composite contains a photobleaching agent that absorbs incident curing radiation and is bleached by the incident radiation.

The invention is directed toward producing photocurable polymeric composites such as coatings, moldings, optical composites, lightweight and high strength composites, biomedical composites, and dental composites. The invention is further directed toward producing a composite and curing lamp (device) system whereby the two are connected by a feedback loop and are able to improve the curing efficiency and also the performance of the composite, and to indicate that the composite is sufficiently cured.

Terms and Definitions:

Photopolymerization or photopolymerizable, as used herein, refers to a process, or materials, in which monomers, oligomers or pre-polymers (hereafter collectively referred to as resins) are polymerized or cured using electromagnetic radiation, such as X-rays, electron beams, and ultraviolet and actinic light. Such materials are hereafter referred to as “light curable”.

Luminescence (or luminescent) as used herein refers to fluorescence and/or phosphorescence.

Luminescent or phosphor materials (collectively luminophores) as used herein, refer to any organic, inorganic or organometallic material capable of absorbing a portion of the incident curing radiation (a first wavelength) and converting it to radiation of a second wavelength. Examples of such materials are further discussed below.

The term index matched as used herein refers to two or more materials that have the same or about the same refractive index.

The term bleach is used to indicate that the agent changes color so that it no longer absorbs the incident radiation

Photobleaching agent, as used herein, refers to a material that absorbs light of a given wavelength, but, as a result of the absorption, is bleached or chemically reacted, so that as it is exposed its absorption is progressively decreased.

A photodetector as used herein is any device that senses, detects or responds to incident light or other electromagnetic radiation in a measurable way.

The invention provides a device and a photo-composite system comprising a light curable resin and at least one luminophore capable of absorbing a first wavelength of light and producing light of a second wavelength, and a curing lamp comprising a light source producing the first wavelength and a detector capable of detecting the second wavelength of light to produce a signal.

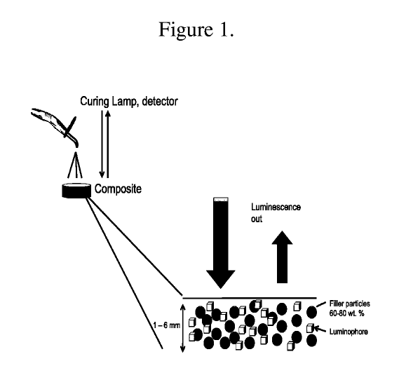

In a preferred embodiment illustrated in FIG. 1, the composite contains a photopolymerizable/curable monomer, oligomer or prepolymer (together referred to as resins). The composite may also contain fillers or other performance materials and addenda as necessary. The composite contains a luminophore, most preferably a phosphor or luminescent material, that is capable of absorbing a portion of the incident radiation of the curing lamp, and emitting a portion of the incident radiation at a second wavelength of light. In a particular embodiment, the luminophore, most preferably a phosphor or luminescent material, absorbs ultraviolet or blue light. In a particular embodiment the luminescent or phosphor material emits at a wavelength between about 450-1000 nm, most preferably between about 550 to 900 nm. The emission intensity of the phosphor is directly proportional to the intensity of incident radiation.

The light curable resin may be selected from any photopolymerizable molecule, monomer, oligomer, or prepolymer (hereafter light curable resins). Particularly preferred light curable resins suitable for use in the application of the invention include hardenable organic materials having sufficient strength, hydrolytic stability, and nontoxicity to render them suitable for use in the oral or in vivo environment. Examples of such materials include acrylates, methacrylates, urethanes, carbamoylisocyanurates, epoxides, and mixtures and derivatives thereof. One class of preferred hardenable materials includes materials having polymerizable components with free radically active functional groups. Examples of such materials include monomers having one or more ethylenically unsaturated group, oligomers having one or more ethylenically unsaturated group, polymers having one or more ethylenically unsaturated group, and combinations thereof. In the class of hardenable matrix resins having free radically active functional groups, suitable light curable components for use in the invention contain at least one ethylenically unsaturated bond, and are capable of undergoing addition polymerization. Such free radically ethylenically unsaturated compounds include, for example, mono-, di- or poly-(meth)acrylates (i.e., acrylates and methacrylates), such as, methyl (meth)acrylate, ethyl acrylate, isopropyl methacrylate, n-hexyl acrylate, stearyl acrylate, allyl acrylate, glycerol triacrylate, ethyleneglycol diacrylate, diethyleneglycol diacrylate, triethyleneglycoldimethacrylate, 1,3-propanediol di(meth)acrylate, trimethylolpropane triacrylate, 1,2,4-butanetriol trimethacrylate, 1,4-cyclohexanediol diacrylate, pentaerythritol tetra(meth)acrylate, sorbitol hexacrylate, tetrahydrofurfuryl(meth)acrylate, bis[1-(2-acryloxy)]-p-ethoxyphenyldimethylmethane, bis[1-(3-acryloxy-2-hydroxy)]propoxyphenyldimethylmethane, ethoxylated bisphenol A di(meth)acrylate, and trishydroxyethyl-isocyanurate trimethacrylate; (meth)acrylamides (i.e., acrylamides and methacrylamides), such as (meth)acrylamide, methylene bis-(meth)acrylamide, and diacetone (meth)acrylamide; urethane (meth)acrylates and the bis-(meth)acrylates of polyethylene glycols. Other suitable free radically polymerizable compounds include siloxane-functional (meth)acrylates and fluoropolymer functional (meth)acrylates. Mixtures of two or more free radically polymerizable compounds can be used, if desired. Other matrix materials or polymers may also be incorporated. Examples of other useful matrix polymers include natural and synthetic biopolymers, such as peptides, proteins, gelatin, poly(lactic acid), poly(glycolic acid), poly(caprolactone), chitosan and its derivatives, alginates, starches and the like.

The composites of the invention typically contain a photoinitiator that is capable to absorb the incident (curing) radiation of a first wavelength and to initiate the polymerization/curing reaction. The photoinitiator may optionally be combined with a sensitizer or accelerator. The choice of photoinitiator may be dependent upon the wavelength of the curing radiation. For X-ray or electron beam radiation, a photoinitiator is not typically required since these high energy wavelengths may directly initiate polymerization. For ultraviolet curing, the photoinitiator is typically selected so that it absorbs energy between about 180-450 nm. For blue light curing, the photoinitiator is typically selected so that it absorbs energy between about 400-500 nm. Examples of suitable UV and visible photoinitiators are those sold under the tradename Irgacure® and Lucirin® (BASF Corp. Charlotte, N.C.) or under the tradename Darucor® (Ciba Specialty Chemicals). It is preferred that the photoinitiator is a blue light photoinitiator that is photobleachable. For dental or medical applications, it is preferred that the photoinitiator is camphorquinone or TPO (2,4,6-trimethylbenzoyldiphenylphosphine oxide). It is further preferred that the camphorquinone is used together with a polymerization accelerator such as an amine, or any other molecule capable of accelerating the reaction. An example of a suitable accelerator for practice of the invention is ethyl-4-N,N-dimethylamino benzoate.

The luminophores of the invention may be selected from any organic, inorganic or organometallic materials capable of absorbing a portion of incident curing radiation, also referred to herein as a first wavelength of light, and converting it to radiation of a second wavelength of light. It is preferred that the second wavelength is greater than that of the incident radiation. Particularly preferred materials are phosphors that are white, yellow or neutral in color, and are capable to absorb ultraviolet and blue light and convert it to light having a wavelength between about 450 to 2000 nm, most preferably from about 500 to 1000 nm. Useful examples of such materials are yttrium aluminum garnets, silicates, sulfides and nitrides, rare earth materials commonly used in the LED lighting industry. Luminophores can be further classified as fluorophores or phosphors, depending on the nature of the excited state responsible for the emission of photons. However, some luminophores cannot be classified as being exclusively fluorophores or phosphors. Examples include transition metal complexes such as tris(bipyridine)ruthenium(II) chloride, whose luminescence comes from an excited (nominally triplet) metal-to-ligand charge transfer (MLCT) state, which is not a true triplet-state in the strict sense of the definition; and colloidal quantum dots, whose emissive state does not have either a purely singlet or triplet spin. Most luminophores consist of conjugated pi-systems or transition metal complexes. There are also purely inorganic luminophores, such as zinc sulfide doped with rare earth metal ions, rare earth metal oxysulfides doped with other rare earth metal ions, yttrium oxide doped with rare earth metal ions, zinc orthosilicate doped with manganese ions, etc.

In a particularly preferred embodiment, the luminophore is selected from a near infrared (NIR) emitting material that emits between about 700-2000 nm. This is preferred because the phosphor emission will not be observed in the visible light spectrum, where such emission could be visible to the human eye. Examples of preferred luminophores are HTY560, LWR 6931, PTIR 790/F, PTIR 1070, indocyanine green (ICG) and cyanine dyes known to be efficient emitters in the NIR.

It is preferred that the luminophores have a high efficiency of converting incident curing radiation of a first wavelength of light to a second wavelength of light. This is preferred because the greater the efficiency of conversion, the less the amount of the luminophore that will be required to be contained with the composite. The luminophore potentially may adversely affect other aspects of the composite including its color, appearance and physical properties.

It is preferred that the loading of the selected luminophore within the composite is less than 10 weight percent, more preferably less than 1 weight percent, and most preferably less than 0.1 weight percent. For highly efficient luminophores the loading can be reduced to less than 500 ppm (or 0.05 weight percent).

Other examples of phosphors that may be suitable in the invention are optical brighteners such as Uvitex OB (BASF Corp.), titanium dioxide, zinc oxide and doped versions of zinc oxide. Optical brighteners are commonly employed in the dental industry whose fluorescence or luminescence can be used to provide whitening.

The light curable coatings or composites of the invention may contain addenda such as flow agents, thickening agents, coating agents, surfactants and performance agents that improve the manufacturability of the article, or improve the physical properties of the final composite or coating. In the coating industry, it is common to add performance addenda that improve adhesion, scratch resistance, hardness and durability of the article. It is preferred that such addenda do not substantially absorb the curing radiation, since the absorbance by addenda may adversely affect the polymerization rate.

There are several methods known within the industry to counteract the absorption of curing radiation by addenda. The first is to include nanoparticles within the coating or composite formulation. Because of their small size (less than about 100 nm), nanoparticles often may not absorb or scatter the curing radiation and hence may be transparent to both the curing, emitted and visible wavelengths. A second method to control this problem is to match the refractive indices of the uncured resin and performance addenda materials. If the addenda have the same, or nearly the same, refractive index as the resin then the curing radiation is not significantly scattered by the performance addenda. This is especially important in the dental industry where the filler loadings are remarkably high (typically about 80 weight percent in order to obtain hardness comparable to teeth). Further the refractive index is matched to provide a composite (tooth restoration) that is aesthetically appealing, attempting to recreate the optical translucency and visual brilliance of natural teeth. It is preferred that the refractive index difference between the resin and fillers is not greater than 0.05, and more preferably not greater than 0.025.

A wide variety of dental filler materials are available to aid formulators in achieving such properties. The materials, useful for the purpose of practicing the instant invention include silica-alumina, silica-titania, silica-zirconia, and silica rare earth mixed oxides as described in H. Suzuki et al. “Refractive index adjustable fillers for visible light cured dental resin composites: preparation of TiO₂—SiO₂ glass powder by the sol gel process.” J. Dental Research. 883 (1991) and in U.S. Pat. Nos. 4,217264, 4,503,169, 4,764,497, 5,856,374, 6,572,693, 6,730,156 and 8,617,306, all incorporated herein by reference in their entirety. Also useful for practice of the invention are refractive index controlled glasses, sometimes referred to as dental glasses, and exemplified most typically by barium glass or strontium glasses, available form Schott Corp. Still other useful materials are nanoparticles or nanoparticle dispersions as described in U.S. Pat. Nos. 5,609,675, 6,060,830, 6,572,693, 6,899,948 and Japanese Patent Kokai JP07-291817, all incorporated herein by reference in their entirety. Still other useful materials are fumed, colloidal or precipitated silicas, aluminas, and radiopaque materials such as zirconia, nano-zirconia, ytterbium fluoride and yttrium fluoride.

Composites may often contain more than one performance addenda (fillers), over even combinations of many fillers. In this case the primary filler is the filler that is used in the greatest quantity. It is preferred that both the resin and the primary filler have a refractive index between about 1.44 to 1.55, and more preferably about 1.49 to 1.54.

The photobleaching agent may be a photoinitiator or any dye or absorbing material capable of being bleached by the incident curing radiation. It is preferred that the photobleaching agent absorbs UV or blue light, is yellow or pale yellow in color, and is photobleachable in the same time frame as the polymerization process. That is to say that if the polymerization requires a ten second exposure to curing light, then the photobleachable agent should be bleached to about 75% of its initial optical density in ten seconds, more preferably to about 50% of its initial optical density, and most preferably to about 25% of its initial optical density in ten seconds. This is preferred because residual color left as a result of incomplete photobleaching may adversely impact the aesthetic quality of the composite. A preferred photobleaching agent is camphorquinone.

The curing lamp (or curing radiation source) may be selected from any X-ray, electron beam, ultraviolet or visible light emitting source. In the application of the invention in the coatings industry, it is preferred that the curing radiation is from an ultraviolet source. In the medical or dental industry, it is preferred that the curing lamp is a long wave ultraviolet or blue light emitting source. A variety of dental curing lamps are available commercially including quartz tungsten halogen lamps, plasma arc lamps, argon ion lasers and light emitting diode (LED) lamps (both single wave and polywave LED). The invention described herein may be configured to work with any of these lamps. One skilled in the art may select the appropriate photoinitiator, photobleaching agent and phosphor after consideration of the emittance wavelength and emittance power of the lamp. It is preferred, for dental applications, that the lamp is selected from a blue emitting LED source as such lamps have excellent power stability and are safer for the patient.

The detector may be selected from a charge coupled device (CCD), c-MOS, photodiode detector, bolometer or microbolometer, reversed biased LED, photomultiplier, photoresistor or any detector capable of detecting the emitted light from the luminophore(s). There may be employed one, two or more detectors. The detector may be placed such that it is in the path of the emitted light, or alternatively a light channeling device such as a fiber optic cable or lens may be employed to channel the emitted light to the detector. There may be employed one, two or more light channeling components.

The light reaching the detector is passed through an optical filter such that the filter excludes as much as possible of the curing radiation. This can be accomplished by using an optical filter such as a long pass or notch filter (referred to herein, collectively, as cut-off filters). The filter may be placed anywhere along the light path of the emitted light, for example, directly in front of the detector, or anywhere along the path of the fiber optic cable. The filter is selected such that it is opaque to the curing radiation but is transparent to, or passes, as much as possible of the emitted radiation from the luminophore. The optical filter should remove greater than 90% of the curing radiation, more preferably greater than 99.99% of the curing radiation. This reduces interference within the detector and produces a signal with a high signal to noise ratio. It is preferred that the cut-off filter is a long pass filter with a cut-off wavelength greater than 550 nm, and more preferably greater than 640 nm. The signal may be voltage or current or any other convenient form of conversion signal.

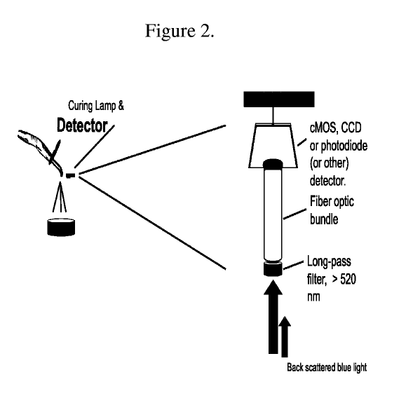

FIG. 2 shows an embodiment of the invention, wherein the detector is shielded from the back scattered curing light by an optical filter, here, a cut-off filter. The cut-off filter allows emitted light from the phosphor to pass through, but is absorbing to, and blocks the curing light. In this manner, only the emitted light is detected. The cut-off filter may be selected by one skilled in the art dependent upon the precise wavelength, or light spectrum, of both the incident curing light and the phosphor. In a particular embodiment, the cut-off filter is optically glued to a fiber optic bundle that serves as a light guide to guide the emitted light to a detector. The detector may be a photodiode, c-MOS, charge coupled device (CCD) or any detector capable of detecting the emitted light from the composite and producing a signal such as a current or voltage signal. In a particular embodiment, it is preferred that the detector is positioned as close to the tip of the curing lamp as possible.

In another embodiment, the inventions provides a device-composite system comprising a dental device-composite system comprising a light curable dental composite comprising a resin, at least one luminophore capable of absorbing a first wavelength of light and producing light of a second wavelength, and one or more fillers, optical brighteners, and photoinitiators, and a dental curing lamp comprising a light source of a first wavelength and a detector capable of detecting the second wavelength of light, wherein the light of a second wavelength is detected by the detector, preferably as close as possible to the light exit position of the dental lamp.

In the application of photocurable resin based composites in dentistry, the curing lamp is preferably a hand-held device. For curing purposes, it is typically held above the uncured composite at a distance (d), and for a time (s) that is arbitrarily selected by the operator and is believed to be sufficient to cure the composite (based upon the manufacturer's recommendations). However, there are many different types of dental lamps, with varied radiant spectrums and radiant power densities. The distance (d) is prone to operator error and the lamp tip or light exit position may be held within a few millimeters of the target, or its position may vary to as much as about 10 mm dependent upon the operator. This variation is highly significant since the radiant intensity of the curing light decreases as 1/d². This means that as the distance doubles the radiant power reaching the target decreases by a factor of four. The instant invention described herein may directly compensate, and correct, for many user variations since the light emitted from the luminescent composite (and the lamp) are governed by the same physical laws.

It is preferred that the light is collected and detected as close to the tip or light exit position of the lamp as possible since this allows for correcting the cure time based upon the position of the lamp tip or light exit position. For example, if the lamp tip and detector are moved closer, both cure irradiance and emission irradiance increase and the device can properly predict that the curing period is shorter. Likewise, if the lamp is held at a distant position, then both decrease and the device can predict that the curing must continue for a longer time. The light may be collected at the tip or light exit position by placing the detector at this position, or alternatively by placing one or more fiber optic cables or bundles at this position to collect the light, and to provide a conduit so that the light may reach the detector.

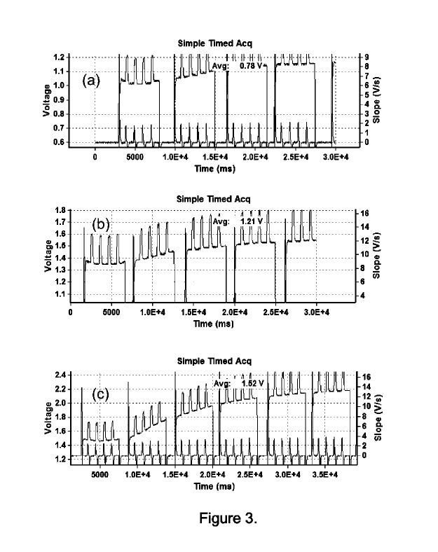

In a particular embodiment, the composite and detector are connected via a feedback loop such that the detector indicates that the composite has absorbed sufficient incident radiation to cure the composite. To create such a feedback loop, electronic hardware is employed to record the luminescence output versus time, and software is employed to examine the data and to predict cure. Referring to the data contained in FIGS. 3-5, in each case it is observed that the signal increases rapidly on initial exposure and then levels off after a time period dependent upon the thickness, volume and shade of the composite. In FIGS. 3-5, the steady state luminescence (i.e., leveling off) can be understood to indicate cure, since this is the point at which the photobleaching and refractive index change are complete. Therefore the device system of the invention can be designed and programmed to examine real time data to recognize steady state luminescence. After the steady state is detected the device may simply indicate to the user, by any visual or audio means, that cure is complete. Alternatively, the device may simply turn itself off, i.e., turn the lamp off.

Applications contemplated for the invention are not limited to the dental or medical industry as described in detail above. Light curable composites are used in a variety of industries, including paints & coatings, printing, including 3D printing, and in the composites industry (aerospace and medical composites). For all of these industries, effective and efficient curing is an important issue, and real time information regarding the incident curing radiation may directly lead to manufacturing efficiency, product quality and cost improvement. A particularly important industry for application of the invention is the roll coating industry. In this industry, light curable resins are applied to a substrate that is moving, often rapidly at as much as several thousand feet per minute. The moving coating is then passed under curing lights to effect polymerization. The curing lights are typically extremely high powered UV lights that require extensive shielding and external cooling.

Although the distance (d) and time (s) may be easily fixed by an engineer in such industrial systems, failed coatings due to insufficient cure are still the major cause of product failure. This is because the high energy lamps wear and their output decreases substantially over time. The invention described herein can be configured or optimized by one skilled in the art to create a device-composite system that monitors incident curing radiation in real time. It should be understood that, in this application, it is not necessary or desirable to place the detector at the light tip, but rather the detector, or multiple detectors, can be placed directly above or below the coating within the path of the incident curing radiation.

### EXAMPLES

### Materials and Methods

HTY560 is a green emitting phosphor purchased from Phosphortech Corp. with a mean particle diameter of about 10 microns. The excitation wavelength recommended range is 440-490 nm and the emission is centered at 560 nm with an emission tail that extends to about 780 nm.

PTIR790/F is a near infrared emitting phosphor purchased from Phosphortech Corp. with a mean particle diameter of about 4.0 microns. The excitation wavelength is between 400-500 nm and the emission bands are at about 710, 790 and 820 nm.

LWR6931 is a red emitting phosphor that was purchased from Intematix Corp. with a mean particle diameter of about 14 microns. It excitation wavelength is between about 200 to 640 nm and its emission is centered at 670 nm.

The curing lamp used for all examples was a Kerr DemiUltra LED with blue light emission centered at about 470 nm and a radiant power of 1100 mW/cm². The light exit position was the tip.

The light detector was an LEX-100 light measurement sensor purchased from EMX industries. A 1.5 mm fiber optic cable was employed to guide the light to the detector. Long pass filters OG570 and RG645 were purchased from Schott Inc. and cut to the desired size.

### EXAMPLE 1

A composite paste was prepared as follows: 35.0 g of dental filler consisting of a silica-zirconia mixed-oxide prepared as described in U.S. Pat. No. 4,503,169 with refractive index 1.53, 15.0 g of a dental resin that contained the activation agent camphorquinone and 0.0112 g of HTY560 phosphor were mixed thoroughly in a centrifugal mixer. The paste was then packed into a stainless steel washer having a diameter of 5.0 mm and a specimen thickness of 1 mm. The specimen was then mounted beneath a dental lamp with the tip of the lamp held at 1 cm distance from the specimen. To collect the emitted phosphor light, a 1.5 mm fiber optic was mounted adjacent to the lamp tip and directly above the specimen. The opposite end of the fiber optic cable was mounted to a long pass filter, Schott RG645, that filtered out all wavelengths below 645 nm. The filter was mounted directly in front of the LEX-100 detector. The dental lamp was set for five second pulses. The detector voltage (luminescence output) was collected throughout each exposure at 160 millisecond intervals.

### EXAMPLE 2

Example 2 was performed identically to Example 1 above except that the specimen thickness was 2 mm.

### EXAMPLE 3

Example 3 was performed identically to Example 1 above except that the specimen thickness was 3 mm.

The data collected for examples 1-3 are shown in FIG. 3. FIG. 3 shows the luminescence output versus time for specimens of thickness 1 mm (a), 2 mm (b) and 3 mm (c), respectively. The exposures were done at five second intervals and repeated five to six times. The voltage signal is pulsed due to pulsations inherent to the dental lamp. The data indicate that the luminescence output of the composite (the upper curve) changes during exposure, initially increasing but leveling off after a short time. The time at which the signal levels off increases with increasing sample thickness. The data indicate that the luminescence output can be used to measure or integrate the total light flux reaching the specimen, and is dependent upon time and specimen thickness. The interpretation of the signals observed with respect to indicating cure of the composite(s) is explained vide supra.

### EXAMPLE 4

Example 3 was performed identically to Example 1 above except that the specimen diameter was doubled (to 10 mm) and the thickness was 2 mm

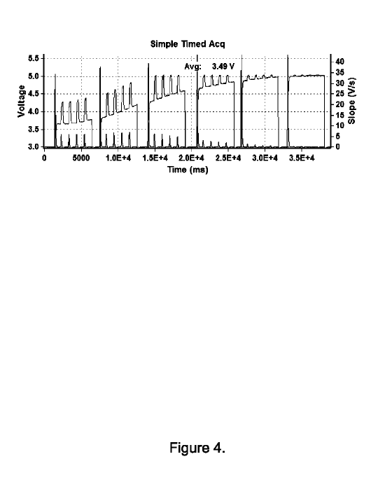

The data of FIG. 4 show that the overall luminescent signal increases (upper curve) substantially for a larger specimen volume, and further that the signal shows the same characteristic increases in luminescence but leveling off after a short time.

### EXAMPLE 5

A composite paste was prepared as follows: 3.060 g of Sonicfil-2 (lightest shade) commercial dental composite (Kerr Corporation), 1.79 mg of PTIR-790/F Phosphor and 1.0 g acetone were added into a brown glass vial. The cap was closed and the contents were sonicated for 30 minutes until a homogeneous slurry was obtained. The acetone was then removed overnight at 50° C. until no further change in sample weight could be detected. The final paste contained 530 ppm of the NIR phosphor. The paste was then packed into a stainless steel washer having a diameter of 5.0 mm and a thickness of 2 mm. The specimen was then mounted beneath a dental lamp with the tip of the lamp held at 1 cm distance from the specimen. To collect the emitted phosphor light, a 1.5 mm fiber optic was mounted adjacent to the lamp tip and directly above the specimen. The opposite end of the fiber optic cable was mounted to a long pass filter, Schott RG645, that filtered out all wavelengths below 645 nm. The filter was mounted directly in front of the LEX-100 detector. The dental lamp was set for five second pulses. The detector voltage (luminescence output) was collected throughout each exposure at 160 millisecond intervals.

### EXAMPLE 6

Example 6 was prepared and performed identically to Example 5 above except that the darkest shade of commercial SonicFil-2 was used and the final NIR phosphor (PTIR-790/F) concentration was 526 ppm.

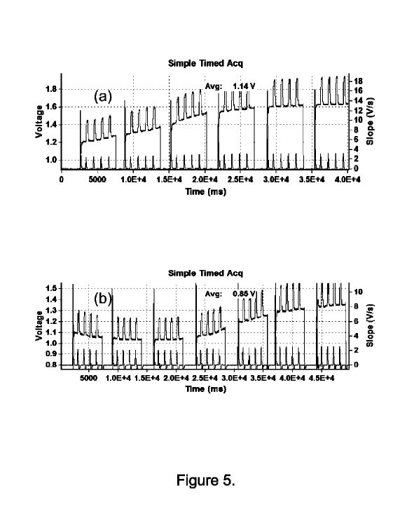

The data collected are shown in FIG. 5. The data of FIG. 5 show that the luminescent output versus time from both (a) unshaded and (b) dark shade SonicFil-2 behaves in a stepwise manner as was observed for previous examples. The data may be understood by reference to the following theory or explanation.

The explanation or theory herein is provided to increase understanding of the invention but is not meant to limit the invention in any way. For all of FIGS. 3-5, the data collected indicate increasing luminescence output of the composite as it is exposed to the curing (blue light) radiation. The luminescence output increases initially, but then levels off after a time interval that is dependent upon the thickness (FIG. 3), volume (FIG. 4) and shade (FIG. 5) of the specimen that is being cured. During irradiation of the specimen, the incident curing light is initially absorbed by the photoinitiator (camphorquinone), which decomposes into free radicals that in turn initiate polymerization of the resin monomers. It is well known that camphorquinone photobleaches during this process (see S. Asmusen, G. Arenas, W. D. Cook and C. Vallo, “Photobleaching of camphorquinone during polymerization of dimethacrylate based resins”, Dental Materials 25, 1603-1611 (2009)). Camphorquinone efficiently absorbs blue light (the curing radiation) but progressively absorbs less curing radiation as it photobleaches. Therefore, during the curing process, the curing radiation progressively penetrates deeper into the specimen. This in turn allows more excitation light to reach the phosphor and hence the emission intensity of the phosphor increases as the camphorquinone is photobleached. After all of the photoinitiator is consumed, the luminescence emission reaches a steady state. In addition, there is a second, and equally important, physical process that occurs during photopolymerization. As the monomers are converted into a polymer, the refractive index of the composite increases (mainly due to the volume change of the polymer versus the monomers). As the refractive index of the polymeric portion of the composite changes, there occurs a mismatch in the refractive index of the filler materials compared to that of the polymer. As the refractive index mismatch increases (during cure), light is increasingly scattered within the composite, this increases the overall path length of the curing light with the composite and hence increases light absorption by the phosphor, hence increasing its emission intensity. The progressively increasing scattering may also increase the amount of light back scattered to the detector (also increasing the emission signal). At the completion of polymerization, the refractive index no longer changes and the emission intensity reaches a steady state.

1. A device-composite system comprising a light curable resin and a luminophore capable of absorbing a first wavelength of light and producing light of a second wavelength, and a curing lamp comprising a light source producing said first wavelength of light and a detector capable of detecting said second wavelength of light to produce a signal.

2. The device-composite system of claim 1 further comprising at least one photobleaching agent.

3. The device-composite system of claim 2, wherein the at least one photobleaching agent is a photoinitiator.

4. The device-composite system of claim 3, wherein the photoinitiator is camphorquinone or 2,4,6-trimethylbenzoyldiphenylphosphine oxide (TPO).

5. The device-composite system of claim 1, wherein a cut-off filter that is capable of shielding the detector from the first wavelength of light is placed between the light curable resin and the detector.

6. The device-composite system of claim 5, wherein the cut-off filter is a long pass filter with a cut-off wavelength greater than 550 nm.

7. The device-composite system of claim 1, wherein the luminophore is a phosphor.

8. The device-composite system of claim 1, wherein the luminophore is a near infrared phosphor.

9. The device-composite system of claim 1, wherein the composite contains less than 1 wt. % luminophore.

10. The device-composite system of claim 1, wherein the curing lamp is selected from a UV or visible light curing lamp.

11. A dental device-composite system comprising a light curable dental composite comprising a resin, at least one luminophore capable of absorbing a first wavelength of light and producing light of a second wavelength, and one or more material selected from the group consisting of fillers, optical brighteners, and photoinitiators, and a dental curing lamp comprising a light source of a first wavelength and a detector capable of detecting said second wavelength of light, wherein the light of a second wavelength is detected by the detector as close as possible to the light exit position of said dental lamp.

12. A dental device-composite system of claim 11, wherein the filler is index matched to the dental resin.

13. A dental device-composite system of claim 11, wherein each of the light curable resin and primary filler have a refractive index between 1.44 and 1.55.

14. A dental device-composite system of claim 11, wherein each of the light curable resin and primary filler have a refractive index between 1.49 and 1.54.

15. A dental device-composite system of claim 11, wherein fiber optic cables are placed at the tip of the dental curing lamp.

16. A dental device-composite system of claim 11, wherein the dental curing lamp is a hand-held device and the detector is housed within the lamp.

17. A dental device-composite system of claim 11, wherein the dental curing lamp is a blue LED lamp.

18. A dental device-composite system of claim 11, further comprising a cut-off filter with a cutoff wavelength greater than 550 nm located between said light curable resin and said detector.
